(19) United States
(12) Patent Application Publication
(10) Pub. No.: US 2025/0237612 A1
(43) Pub. Date: Jul. 24, 2025

(54) FLUID SAFETY DEVICE FOR DETECTING AND MONITORING SPOILAGE OF PERISHABLE FLUIDS

(71) Applicant: MOHAMMED AL-MANA COLLEGE FOR MEDICAL SCIENCES, Dammam (SA)

(72) Inventor: Fardus Abdullah ALJUNIBI, Dammam (SA)

(73) Assignee: MOHAMMED AL-MANA COLLEGE FOR MEDICAL SCIENCES, Dammam (SA)

(21) Appl. No.: 18/419,637

(22) Filed: Jan. 23, 2024

(51) Int. Cl. G01N 21/80; G01N 21/29; G01N 21/77; G01N 33/14

(52) U.S. Cl. CPC G01N 21/80; G01N 21/293; G01N 33/14; G01N 2021/7759; G01N 2201/024

(57) ABSTRACT

A fluid safety device for quick detection of spoilage in food products. The detection method is based on the presence of various contaminants that serve as indicators of food spoilage. The fluid safety device is an easy-to-use, cost-effective device for monitoring the quality of perishable food products, especially fluids, and comprises a reaction pad separated into sections wherein multiple contaminants in the fluid can be tested simultaneously.

### STATEMENT OF ACKNOWLEDGEMENT

Support and facilities provided by the Mohammed Al-Mana College of Heath and Sciences is gratefully acknowledged.

### BACKGROUND

### Technical Field

The present disclosure is directed to a fluid safety device, particularly a fluid safety device for detecting and monitoring spoilage of perishable fluids.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Spoilage of food occurs when it is left unused for an extended period. One of the reasons for food spoilage is microbial activity occurring in the food that releases by-products that change the odor, color, taste, or smell of the food. Sometimes, the food products have expiry dates mentioned on their packaging, and even if the food product does not show any signs of spoilage, the consumer of a product past its defined expiry date product throws it away. Consumers also refrain from buying goods near their expiration dates. If not sold, these goods return to the manufacturers and sellers, who may repack them with new expiration dates. Such goods, if spoiled, adversely affect the health of the consumers.

The problem of food spoilage is more pronounced in liquids due to their water content. Liquid foods, especially milk-based liquids, have a relatively shorter shelf life; hence, consumers are advised to consume them long before their expiration dates. One example of a popular milk-based product is baby formula. Parents have used baby formula to feed their children for many centuries. It's an excellent substitute for breast milk because it has all the nutrients a baby needs to grow properly. Baby formula comes in various forms, such as ready-to-serve liquids, powders, or concentrates, and each form has a unique preservation method. However, such preservation methods can work only for a short time. The nutrients in the baby formula start degrading after a certain period, and then, the baby formula cannot be given to the infant. Sometimes, spoiled liquid food products are thrown away if there is a change in their color, texture, or smell, even if no microbes or toxins are present. This leads to the wastage of large quantities of food, resources, and money.

Several devices and detection methods have been developed to detect food spoilage and prevent food waste. Devices based on chemicals and biosensors are being developed that can detect the quality of food based on various parameters like the presence of certain compounds, the release of gaseous products in the food, and changes in color, odor, or texture. Recently, sensors based on screen-printed electrodes (SPE) have been designed to evaluate food safety and freshness. Such sensors are biosensors and detect food spoilage based on immunoassays or detection of biogenic amines in the food samples [See: Ricarda Torre, Estefanía Costa-Rama, Henri P. A. Nouws and Cristina Delerue-Matos Screen-Printed Electrode-Based Sensors for Food Spoilage Control: Bacteria and Biogenic Amines Detection. Biosensors 2020, 10 (10), 139)]. Nanomaterials are also being explored to design sensors that detect or monitor food spoilage based on their properties. These sensors can be placed directly in the smart packaging of food products to detect spoilage or adulteration [See: Zahra Mohammadi, Seid Mahdi Jafari Detection of food spoilage and adulteration by novel nanomaterial-based sensors. Advances in Colloid and Interface Science. Volume 286, December 2020, 102297].

US20040115319 discloses a device for detecting food-borne pathogens and spoilage wherein the device has a gas-permeable sensor housing, and a pH indicator placed inside the housing. The pH indicator detects a change in the gaseous bacterial metabolite concentration, indicative of bacterial growth. US20040115319 also discloses smart packaging for packing food products wherein the device can be placed within the packaging of the food product, and a change in the pH indicates the presence of pathogens in the food product.

Each of the sensors, as mentioned above, and devices is time-consuming and expensive. Additionally, existing devices based on sensors have their limitations. For example, they are primarily useful for solid food products. Biosensor-based detection methods need to be stored in specific storage conditions to function effectively. Further, most of the sensors for detection can selectively identify only one source of food spoilage. Since the cause of food spoilage is not always predictable, one may need more than one type of sensor to detect food spoilage due to various sources. Hence, a need remains for devices that can detect food spoilage caused by multiple sources and are cost-effective, efficient, and valuable to test edible products in liquid form. Accordingly, one object of the present disclosure is to provide food safety devices for fluidic foods capable of monitoring food quality and detecting spoilage while being precise and efficient.

### SUMMARY

In an exemplary embodiment, a fluid safety device is described. The fluid safety device comprises a rectangular prism having a proximal end, a body, and a distal end. The fluid safety device further includes a standard color card. The proximal end comprises a hand grip, and the distal end comprises a reaction pad separated into at least a first section, a second section, a third section, and a fourth section. Each section comprises a detector, and each detector comprises a sensor and a color-changing indicator. The sensor detects a contaminant in a test sample and activates the color-changing indicator.

In some embodiments, the sensor of at least one of the first, second, third, or fourth sections comprises a sulfonephthalein dye. The sulfonephthalein dye is selected from the group consisting of bromophenol blue, bromocresol green, chlorophenol red, bromothymol blue, o-cresol red, and bromocresol purple.

In some embodiments, the sensor of at least one of the first, second, third, or fourth sections is potassium-iodide starch paper.

In some embodiments, each section is separated by a channel comprised of a solid substrate, wherein the channel further comprises at least two axes, and wherein a first axis is arranged perpendicularly to a base of the reaction pad and a second axis is arranged parallel to the base of the reaction pad to cross over the first axis.

In some embodiments, each section is arranged so that at least a side of each section is touching at least a side of another section.

In some embodiments, each section is an absorptive well comprising a sponge material, and the sensor and the color-changing indicator are combined with the sponge material.

In some embodiments, each section comprises a solid base with at least a first layer and a second layer stacked on the solid base. The first layer comprises the sensor and the second layer comprises the color-changing indicator.

In some embodiments, the detector of at least the first, second, third, or fourth section comprises a nanoporous polyacrylonitrile mat as the sensor, and the nanoporous polyacrylonitrile mat is impregnated with a colorimetric substance as the color-changing indicator.

In some embodiments, the sensor of at least one of the first, second, third, or fourth sections comprises a soda lime pad, a hydrophobic membrane, and a pH indicator.

In some embodiments, the hydrophobic membrane is selected from the group consisting of a polymeric microfiltration membrane, a polyvinylidene difluoride (PVDF) membrane, a positively charged nylon transfer membrane, a polytetrafluoroethylene-supported (PTFE) membrane on polypropylene or polyester, a microporous PTFE membrane, and a polypropylene membrane.

In some embodiments, the sensor of at least the first, second, third, or fourth section is comprised of a cellulosic substrate infused with a colorimetric solution.

In some embodiments, the colorimetric solution comprises a colorimetric substance and an alcohol, and the colorimetric substance is selected from the group consisting of phenolphthalein, p-xylenolphthalein, and thymolphthalein.

In some embodiments, the sensor of at least the first, second, third, or fourth section is a blotting paper comprising a triglyceride detecting mixture, and the triglyceride detecting mixture comprises an HCl buffer, an enzyme, a dye, and a diaphorase solution.

In some embodiments, the sensor of at least the first, second, third, or fourth section is a blotting paper applied with a triglyceride detecting mixture, and the triglyceride detecting mixture comprises a solution comprising water, a nonionic surfactant, a detergent, a poly methyl vinyl ether, calcium chloride, Na-ATP, sucrose, and HPC (hydroxypropyl cellulose). The triglyceride detecting mixture further comprises an enzyme, a chromogenic substrate, a horseradish peroxidase mixture, and EDTA.

In some embodiments, the sensor of at least the first, second, third, or fourth section is a filter paper applied with a triglyceride detecting mixture, wherein the triglyceride detecting mixture comprises a dye solution, and the dye solution comprises a lysochrome selected from the group consisting of an amino acid staining azo dye, an aromatic compound containing an azo group, and a diazo dye.

In some embodiments, the sensor of at least the first, second, third, or fourth section is comprised of an octanoyl-CoA substrate.

In some embodiments, the standard color card has a chart of colors which correlate to the presence of the contaminant in a test sample, and a user can determine the presence of the contaminant in the test sample based on a color change of the reaction pad when contacted with a test sample and the standard color card.

In some embodiments, the distal end further comprises a reaction pad well having a bottom surface and four sides that form an enclosure capable of holding a liquid and having an open top, wherein the four sides are the same height as the detectors in each section. The bottom surface of the reaction pad well is integrated into an outer surface of the distal end of the fluid safety device such that the reaction pad well retains a fluid when positioned horizontally.

In some embodiments, the reaction pad well further comprises a retractable cover that seals the enclosure.

In some embodiments, the proximal end further comprises a retractable bipodal support extension wherein the bipodal support extension has a height such that the enclosure is level with a flat surface on which the fluid safety device is disposed, and the distal end of the fluid safety device is oriented upwards at an angle of 10-30° from the axis of the body.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 shows a schematic perspective view of a fluid safety device, according to certain embodiments.

FIG. 2A shows a schematic enlarged perspective view of a distal end of the fluid safety device with enclosures to hold a test sample and a retractable cover to seal the enclosures, according to certain embodiments.

FIG. 2B shows a schematic enlarged perspective view of the distal end of FIG. 2A in which the retractable cover is extended to seal the enclosures, according to certain embodiments.

FIG. 3A shows a schematic side view of the fluid safety device positioned on a flat surface, when not in use, according to certain embodiments.

FIG. 3B shows a schematic perspective view of a proximal end of the fluid safety device with a bipodal support extension coupled thereto, according to certain embodiments.

FIG. 3C shows a schematic side view of the fluid safety device positioned on the flat surface with the help of the bipodal support extension, when in use, according to certain embodiments.

FIG. 4 illustrates a schematic block diagram of a system having the fluid safety device for detecting contaminants in the test sample, according to certain embodiments.

FIG. 5 illustrates a schematic prototype model of the fluid safety device, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, “compound” refers to a chemical entity, whether as a solid, liquid, or gas, and whether in a crude mixture or isolated and purified.

As used herein, “fluid” refers to a substance that can flow and has no fixed shape. The fluid herein is a liquid, gas or gaseous substance.

As used herein, “perishable” refers to a food item which is likely to get spoiled or deteriorate in quality as time progresses.

As used herein, “colorimetric substance” refers to compounds that undergo visible change in color in the presence of another chemical species that alter certain conditions such as pH. The terms “colorimetric substance” and “color changing indicator” are used interchangeably and have the same meaning.

As used herein, “spoilage” refers to deterioration in the quality of food under certain conditions such that the food becomes unsuitable or harmful for consumption.

As used herein, “sensor” refers to a compound or a mixture of compounds and other entities that serve as indicator of the presence of desirable or undesirable compounds or chemical species.

As used herein, “section” refers to individual parts in which a material is divided such that each part of the material can be used for a different purpose.

As used herein, “nanoporous” refers to a porous structure having a pore size of 1-100 nm.

As used herein, “polymer” refers to a substance made of numerous simpler units called monomers.

The present disclosure is intended to include all hydration states of a given compound or formula, unless otherwise noted or when heating a material.

Aspects of the present disclosure are directed to fluid safety devices to detect and monitor spoilage in food products, especially perishable fluids. More particularly, aspects of the present disclosure are directed to fluid safety devices that can simultaneously detect multiple contaminants suspected to be present in the fluids that may have undergone spoilage.

Disclosed herein is a fluid safety device for the detection of spoilage in fluids. The device includes a reaction pad having compounds that react with contaminants that may be present in the fluids to be tested. A chemical reaction between the compounds and the contaminants results in a change in the color of the reaction pad when contacted with the fluid sample. The presence of a contaminant in the fluid sample is analyzed based on a comparison between the color changes in the reaction pad and colors in the standard color chart.

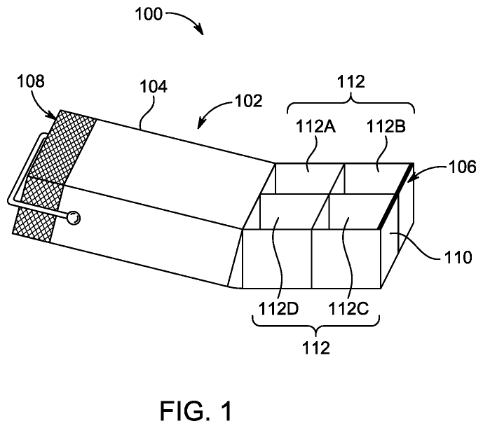

A fluid safety device 100 is illustrated in FIG. 1, according to an embodiment of the present disclosure. The fluid safety device 100 comprises a rectangular prism 102. The rectangular prism 102 may be otherwise defined as a prism having a rectangular cross-section. In some embodiments, the fluid safety device 100 may include a prism having a square cross-section or a cross-section with any other polygon shape known in the art. A body 104 of the rectangular prism 102 has a distal end 106 and a proximal end 108. The distal end 106 of the fluid safety device 100 comprises a reaction pad 110 integrated therewith. Preferably, the reaction pad 110 is separated into a plurality of sections 112. In an embodiment of the present disclosure, the reaction pad 110 includes a first section 112A, a second section 112B, a third section 112C, and a fourth section 112D. The first section 112A, the second section 112B, the third section 112C, and the fourth section 112D are collectively referred to as ‘the sections 112’ and individually referred to as ‘the section 112’ unless otherwise specifically mentioned.

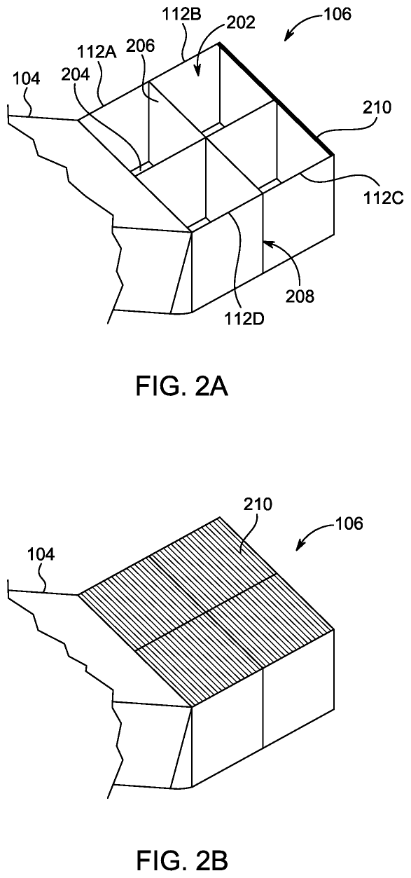

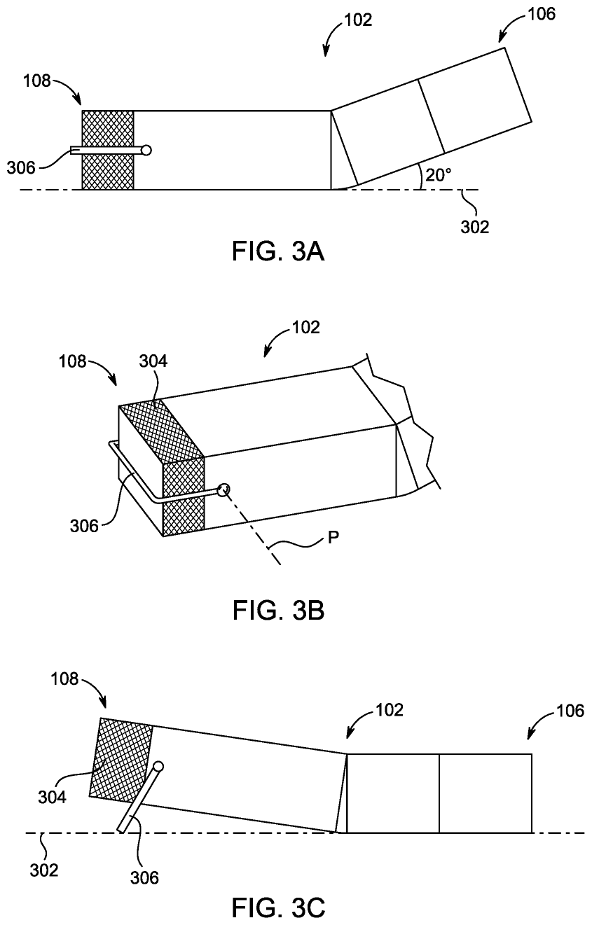

As illustrated in FIG. 2A, the distal end 106 of the rectangular prism 102 includes a plurality of reaction pad wells 202 which may be formed as a well like structure. In an embodiment, each section 112 of the reaction pad 110 may include the reaction pad well 202. The reaction pad well 202 has a bottom surface 204 and sides 206 that form an enclosure capable of holding the test sample. In particular, the reaction pad well 202 includes four sides 206 extending vertically from the bottom surface 204 to form the enclosure with an open top. As such, the reaction pad well 202 is configured to filling with the test sample through the open top. The bottom surface 204 of the reaction pad well 202 is integrated into an outer surface of the distal end 106 of the fluid safety device 100 such that the reaction pad well 202 retains the test sample when positioned horizontally. The outer surface of the distal end 106 of the fluid safety device 100 may be otherwise referred to as a bottom side of the fluid safety device 100 configured to contact with a flat surface 302 (shown in FIG. 3A) of a ground or a test platform such that the fluid safety device 100 may be positioned upright on a surface to hold the test sample in the enclosure.

In an embodiment of the present disclosure, each section 112 of the reaction pad 110 is separated from the other by a channel 208. In one embodiment, the channel 208 comprises at least two axes. A first axis of the channel 208 is arranged perpendicularly to a base of the reaction pad 110 and a second axis is arranged parallel to the base of the reaction pad 110 such that the first axis crosses over the second axis. As such, each section 112 is configured to hold the test sample when the fluid safety device 100 is placed horizontally on the flat surface 302. In an embodiment, the channel 208 is made of a solid substrate.

Each channel 208 may be in fluid communication with one or more of the sections 112. For example, a channel 208 may traverse the length or width of the device in a manner parallel to the base of the reaction pad 110 or perpendicular to the base of the reaction pad 110 such that the channel 208 moves laterally along the axis and also upwards and downwards with respect to the depth of each section 112. Each channel 208 may have a blockage at an end corresponding with the outer walls of the sections 112. In this configuration, wherein a channel 208 crosses the entire length of the reaction pad and moves upwardly from a bottom portion of a first section 112 to an upper portion of another section 112, and the channel 208 has openings that permit fluid communication between the channel 208 and one or more of the sections 112, a sample material may be added to a first section 112 in an amount that is sufficient to cover the reaction pad in the first section up to a point where an opening is present connecting the section 112 to the channel 208. The test sample flows into the first section 112, up to the level of the opening, flows through the opening and up the channel 208 until reaching a second opening into a second section 112. Upon reaching the second opening, the sample material then enters the second section 112. Depending on the number of openings in a particular channel 208, 1, 2, 3, or 4 sections 112 may be exposed to the test sample fluid in sequence, based on the height of the opening between the section 112 and the channel 208 and the presence of an opening from the channel 208 into any particular section 112.

In some embodiments, each section 112 of the reaction pad 110 is arranged in a manner such that at least one side of each section 112 is touching at least one side of another section 112. For example, as depicted in FIG. 2A, one side of the first section 112A touches at least one side of the second section 112B. Similarly, one side of the second section 112B touches at least one side of the third section 112C.

The reaction pad well 202 further includes a retractable cover 210 which is mounted at the top of the distal end 106 that can be extended forward to seal the enclosures in each section 112. As shown in FIG. 2B, the retractable cover 210 that extends over the top of the distal end 106 to seal the enclosure. Sealing the enclosures is desirable to hold the test sample in place when the test sample is poured into the reaction pad well 202. In some embodiments, the retractable cover 210 may be made of an elastomeric material such that the retractable cover 210 may be extended forward to seal the enclosure when required. When the force required to extend the retractable cover 210 is removed, the retractable cover 210 may move backward to its normal position due to an inherent biasing force thereof. In some embodiments, the retractable cover 210 may be made of materials such as plastics or metals, and a retracting mechanism may be used to extend or retract the retractable cover 210 as desired.

As illustrated in FIG. 3A, for better positioning of the fluid safety device 100 on the flat surface 302, the distal end 106 of the fluid safety device 100 is designed in such a way that the distal end 106 is oriented upwards from an axis of the body 104 of the fluid safety device 100. In one embodiment, the distal end 106 is oriented upwards at an angle of 10-30° from the axis of the body 104 of the fluid safety device 100. In some embodiments, the distal end 106 may be oriented upwards at an angle of 15-25° from the axis of the body 104 of the fluid safety device 100. In some embodiments, the distal end 106 may be oriented upwards at an angle of 20° from the axis of the body 104 of the fluid safety device 100. For example, as shown in FIG. 3A, the distal end 106 is oriented upwards at an angle of about 20° from the axis of the body 104 of the fluid safety device 100.

Referring to FIG. 3B, a schematic perspective view of the proximal end 108 of the fluid safety device 100 is illustrated, according to an embodiment of the present disclosure. The proximal end 108 includes a hand grip 304 such that a user can firmly hold the fluid safety device 100 when the fluid safety device 100 is in use. In an embodiment, the hand grip 304 may be provided at a top face, a bottom face and both side faces of the proximal end 108 for proper holding of the fluid safety device 100. In some embodiments, the hand grip 304 may be provided on at least the opposite faces, such as the top and bottom faces or the side faces of the proximal end 108, such that the user may firmly hold the fluid safety device 100 using two fingers. In one example, the hand grip 304 may be a rough or a textured surface defined on the faces of the proximal end 108. In another example, multiple grooves or protrusions may be defined on the faces of the proximal end 108.

The proximal end 108 further includes a bipodal support extension 306 pivotally coupled thereto. As shown in FIG. 3B, the bipodal support extension 306 is a U-shaped bar having free ends pivotally attached to the side faces of the proximal end 108. The bipodal support extension 306 is rotatable about a pivot axis ‘P’. In some embodiments, the bipodal support extension 306 may include a pair of legs pivotally attached to side faces of the proximal end 108. The pair of legs may rotate about the pivot axis ‘P’. The bipodal support extension 306 is retractable and can be extended downwards to provide necessary support to the fluid safety device 100 when placed horizontally on the flat surface 302.

As illustrated in FIG. 3C, the bipodal support extension 306 is mounted at a height such that when the bipodal support extension 306 is extended downwards, the enclosures of the reaction pad well 202 are in level with the flat surface 302 on which the fluid safety device 100 is placed. In other words, the bipodal support extension 306 has a height to position the enclosures in level with the flat surface 302. In some embodiments, the bipodal support extension 306 may be integrated with a locking mechanism to lock the bipodal support extension 306 upon positioning the enclosure on the flat surface 302.

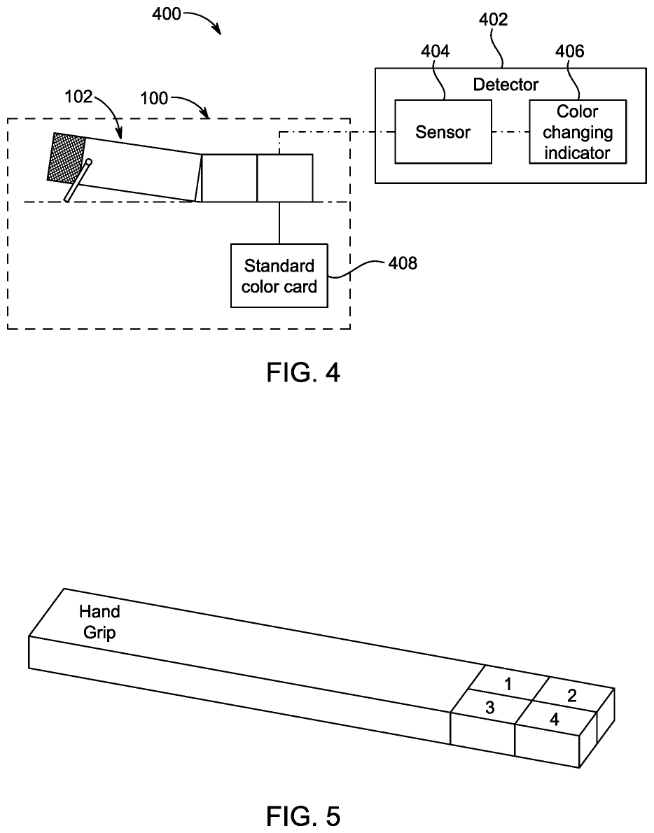

Referring to FIG. 4, a schematic block diagram of a system 400 for detecting contaminants in the test sample using the fluid safety device 100 is illustrated, according to an embodiment of the present disclosure. The system 400 includes a detector 402 disposed in each section 112 of the reaction pad 110. The detector 402 is configured to detect the contaminants contained in the test sample stored in the enclosure defined by each section 112 of the reaction pad 110. The detector 402 includes a sensor 404 for detecting the presence of the contaminants in the test sample. In an embodiment, at least one detector 402 is placed in at least one section 112 in a way that the detector 402 is of the same height as the four sides 206 of each section 112. The detector 402 further comprises a color changing indicator 406 which is activated if the contaminants are present in the test sample. In an embodiment, the sensor 404 may be in electric communication with the color changing indicator 406. As such, the sensor 404 detects the contaminants and/or one or more characteristics such as pH and temperature in the test sample and activates the color changing indicator 406. In one example, the color changing indicator 406 may be a visual indicator which may display different colors in response to the signals received from the sensor 404 based on the concentration of contaminants in the test sample. In another example, the color changing indicator 406 may be an audio indicator which may produce different sounds in response to the signals received from the sensor 404 based on the concentration of the contaminants in the test sample. The fluid safety device 100 further includes a standard color card 408 which may be integrated into a side wall or top surface of the fluid safety device. The standard color card 408 has a chart of colors that correlate to the presence of the contaminants in the test sample. Different colors may be defined based on the concentration of the contaminants in the test sample. Using the standard color card 408, a user can determine the presence of the contaminants in the test sample based on a color change of the reaction pad 110 when contacted with the test sample and the standard color card 408.

In one embodiment of the present disclosure, each section 112 of the reaction pad 110 is an absorptive well comprising a sponge material. In other words, where the enclosures comprise the sponge material, the sections 112 may act as absorptive wells. Further, the sensor 404 and the color changing indicators 406 are combined with the sponge material.

In another embodiment of the present disclosure, the sensor 404 and the color changing indicator 406 are arranged in the form of layers in each section 112 of the reaction pad 110. The section 112 includes a solid base and a first layer comprising the sensor 404 is stacked over the solid base. The second layer comprising the color changing indicator 406 may be placed over the first layer stacked over the solid base.

The sensor 404 acts by detecting the changes in pH levels and the presence of gaseous products, specific proteins, or fatty acid products as metabolites produced by the microbial activities in the food product. The sensor 404 may include various organic or inorganic compounds. In one embodiment, the sensor 404 of the first section 112A, the second section 112B, the third section 112C, or the fourth section 112D may include a dye compound. In some embodiments, the dye compound is a sulfonephthalein dye compound. Sulfonephthalein dyes are very sensitive to pH differences and are used as pH indicators. In certain embodiments, the sulfonephthalein dye may be selected from bromophenol blue, bromocresol green, chlorophenol red, bromothymol blue, o-cresol red, and bromocresol purple. A change in the color of the dye is indicative of a change in the pH level. For example, bromothymol blue changes color from yellow to blue between pH 6 to 9. This dye shows an orange color at pH 5 that changes to yellow and green and finally to dark blue at pH 9. Chlorophenol red has a pH range of 4.8 to 6.7. The dye is yellow around pH 4.8-5 but changes color to violet at pH 6.7. Bromocresol green is yellow at pH below 3.8, and changes color to blue-green above pH 5.4.

In some embodiments, the sensor 404 of the first section 112A, the second section 112B, the third section 112C, or the fourth section 112D may include a potassium-iodide starch paper. Potassium-iodide starch paper can detect the presence of oxidizing agents, including nitrites, peroxides, iodine, and free chlorine. A reaction of potassium iodide with the oxidizing agents results in the production of elemental iodine. The elemental iodine reacts with the starch in the paper to give a blue color.

In some embodiments, the sensor 404 of the first section 112A, the second section 112B, the third section 112C, or the fourth section 112D may include an organic polymer mat. In one embodiment, the organic polymer mat is made of polyacrylonitrile. In a preferred embodiment, the organic polymer mat is a nanoporous polyacrylonitrile mat. Nanoporous polyacrylonitrile mats have high chemical stability and strength and can be used for the detection of gases and gaseous products that may be produced as a by-product of enzymatic or microbial metabolic activities. In some embodiments, the nanoporous polyacrylonitrile mat may be impregnated with a colorimetric substance. The colorimetric substance is a color-changing indicator wherein the color-changing indicator may be a dye. In one embodiment, the colorimetric substance may be selected from bromocresol green, bromophenol blue, and chlorophenol red.

In some embodiments, the sensor 404 of the first section 112A, the second section 112B, the third section 112C, or the fourth section 112D may include a soda lime pad. Soda lime is useful in the detection of gases that may be released in the food product, resulting from the microbial activity in the food product. In one embodiment, a hydrophobic membrane is used along with the soda lime pad as the sensor 404, wherein the hydrophobic membrane provides sufficient surface area for the soda lime to interact with the fluid food sample to be tested. In some embodiments, the hydrophobic membrane may be selected from the group of a polymeric microfiltration membrane, a polyvinylidene difluoride (PVDF) membrane, a positively charged nylon transfer membrane, a polytetrafluoroethylene-supported (PTFE) membrane on polypropylene or polyester, a microporous PTFE membrane, and a polypropylene membrane. In another embodiment, a pH indicator is incorporated in the hydrophobic membrane to enhance the visibility of color change that may occur owing to a change in pH of the fluid sample when soda lime reacts with the metabolite gases such as ammonia, hydrogen sulfide, or carbon dioxide. The pH indicator may be a colorimetric substance, whereas the colorimetric substance may be a pH-sensitive dye. The colorimetric substance may be selected from phenolphthalein, p-xylenolphthalein, and thymolphthalein.

In some embodiments, the sensor 404 of the first section 112A, the second section 112B, the third section 112C, or the fourth section 112D may include a polymeric substrate wherein the polymeric substrate may be infused with a colorimetric solution. The polymeric substrate may be chosen based on its strength, availability, and compatibility with the colorimetric solution. In a preferred embodiment, the polymeric substrate is a cellulose-based substrate. In some embodiments, the colorimetric solution is formed of a colorimetric substance and an alcohol. The colorimetric substance may be a color-changing indicator. In one embodiment, the colorimetric substance is a pH-sensitive dye wherein the pH-sensitive dye is selected from a group consisting of phenolphthalein, p-phenolphthalein, and thymolphthalein. In one embodiment, the colorimetric substance is dissolved in an alcohol to form a colorimetric solution. In some embodiments, the alcohol may be isopropyl alcohol. In a preferred embodiment, the alcohol is ethyl alcohol.

Oxidation of triglycerides is one of the significant parameters for the detection of spoilage in a food product. Triglyceride oxidation occurs when a food product gets spoiled due to various reasons. Under oxidizing conditions, the triglycerides break down to free fatty acids and detecting the amount of triglycerides further helps in determining the quality of the food product to be tested. Accordingly, in some embodiments, the sensor 404 of the first section 112A, the second section 112B, the third section 112C, or the fourth section 112D may include a triglyceride detecting mixture for the detection of triglycerides in the fluid sample. In certain embodiments, the triglyceride detecting mixture is adsorbed on a paper, preferably a blotting paper. In some embodiments, the triglyceride detecting mixture comprises an HCl (hydrochloric acid) buffer, an enzyme, a dye, and a diaphorase solution. In some embodiments, the triglyceride detecting mixture for detecting triglycerides includes a solution of water, a non-ionic surfactant, a detergent, a polymethylvinyl ether, calcium chloride, Na-ATP, sucrose, and HPC (hydroxypropyl cellulose), an enzyme, a chromogenic substrate, a horseradish peroxidase mixture and EDTA.

In some embodiments, the triglyceride detecting mixture for detecting triglycerides includes a dye solution; the dye solution includes a fat-soluble dye that may be dissolved in an alcohol. In one embodiment, the fat-soluble dye is a lysochrome dye. Lysochrome dyes can detect the presence of triglycerides, fatty acids, and lipoproteins owing to their high affinity for fats. In some embodiments, the lysochrome dye is an amino acid staining azo dye. In a preferred embodiment, the lysochrome dye is amido black. In some embodiments, the lysochrome dye is an aromatic compound containing an azo group. In a preferred embodiment, the lysochrome dye is Sudan Red. In some embodiments, the lysochrome dye is a diazo dye wherein the diazo dye is selected from a group consisting of Sudan IV, Sudan III, Oil Red O, and Sudan Black.

In some embodiments, the sensor 404 of the first section 112A, the second section 112B, the third section 112C, or the fourth section 112D may comprise an octanoyl-CoA substrate.

Referring to FIG. 5, a prototype sketch of the fluid safety device 100 is described. As can be observed in FIG. 5, the numbers 1, 2, 3, and 4 correspond to the first section 112A, the second section 112B, the third section 112C, and the fourth section 112D, respectively. The first section 112A includes a pH detector; the second section 112B includes a non-pH detector; the third section 112C includes a fat detector; and the fourth section 112D includes an NH₃ detector. The location of the detectors in the fluid safety device 100 is interchangeable, as may be obvious to a person skilled in the art. When the fluid safety device 100 is brought in contact with a fluid sample to be tested, such as milk, the pH detector detects the change in pH level using suitable dyes like sulfonephthalein dye; the non-pH detector detects the bacterial presence in the milk; the fat detector measures the amount of oxidized fatty acids in the milk and its color change is indicative of the degree of fatty acid oxidation; while the ammonia detector detects for the presence of ammonium compounds in the milk, using a phenolphthalein indicator. Unlike conventional devices, which require the utilization of multiple reaction pads to widen the range of tested compounds, the fluid safety device 100 of the present disclosure obviates the need for the use of multiple reaction pads and is capable of testing multiple compounds (chemicals or microorganisms) in fluids such as beverages, milk, etc., to confirm safe consumption.

The fluid safety device 100 of the present disclosure can detect multiple contaminants simultaneously from a single fluid sample. The fluid safety device 100 may implement one or more sensors 404 depending upon the number and nature of contaminants expected to be present in the fluid sample. For example, the contaminants may be chemical or biological in nature. Also, one sensor 404 may be able to detect more than one type of contaminant in the sample. Accordingly, sections 112 in the fluid safety device 100 may comprise any number of sensors 404 in any combination.

In some embodiments, the fluid safety device 100 comprises a color chart for comparing the color changes occurring in the reaction pad 110 on contact with a fluid sample. The color chart may be a standard color chart with a series of colors. Analysis of a fluid sample based on color comparison determines the quality and freshness of the fluid sample.

Numerous modifications and variations of the present disclosure are possible in light of the above teachings. It is therefore to be understood that within the scope of the appended claims, the invention may be practiced otherwise than as specifically described herein.

1. A fluid safety device comprising:
a rectangular prism having a proximal end, a body, and a distal end; and
a standard color card,
wherein the proximal end comprises a hand grip,
wherein the distal end comprises a reaction pad separated into at least a first section, a second section, a third section, and a fourth section,
wherein each section comprises a detector,
wherein each detector comprises a sensor and a color-changing indicator, and
wherein each sensor is capable of detecting a contaminant in a test sample and activating the color-changing indicator.

2. The fluid safety device of claim 1, wherein the sensor of at least one of the first, second, third, or fourth sections comprises a sulfonephthalein dye,
wherein the sulfonephthalein dye is selected from the group consisting of bromophenol blue, bromocresol green, chlorophenol red, bromothymol blue, o-cresol red, and bromocresol purple.

3. The fluid safety device of claim 1, wherein the sensor of at least one of the first, second, third, or fourth sections is potassium-iodide starch paper.

4. The fluid safety device of claim 1, wherein each section is separated by a channel comprised of a solid substrate,
wherein the channel further comprises at least two axes,
wherein a first axis is arranged perpendicularly to a base of the reaction pad and a second axis is arranged parallel to the base of the reaction pad to cross over the first axis, and
wherein each section is arranged so that at least one side of each section touches at least side of another section.

5. The fluid safety device of claim 1, wherein the reaction pad further comprises a channel,
wherein the channel crosses the entire length of the reaction pad and moves upwardly from a bottom portion of the first section to an upper portion of the second, third, or fourth sections,
wherein the channel has an opening that permit fluid communication between the channel and one or more of the sections,
wherein the test sample is added to the first section in an amount sufficient to cover the reaction pad in the first section up to a point where the opening is present, and
wherein the test sample flows through the opening in the channel into another section in sequence.

6. The fluid safety device of claim 1, wherein each section is an absorptive well comprising a sponge material, and wherein the sensor and the color-changing indicator are combined with the sponge material.

7. The fluid safety device of claim 1, wherein each section comprises a solid base with at least a first layer and a second layer stacked on the solid base, and
wherein the first layer comprises the sensor and the second layer comprises the color-changing indicator.

8. The fluid safety device of claim 1, wherein the detector of at least the first, second, third, or fourth section comprises a nanoporous polyacrylonitrile mat as the sensor, and
wherein the nanoporous polyacrylonitrile mat is impregnated with a colorimetric substance as the color-changing indicator.

9. The fluid safety device of claim 1, wherein the sensor of at least one of the first, second, third, or fourth sections comprises a soda lime pad, a hydrophobic membrane, and a pH indicator.

10. The fluid safety device of claim 9, wherein the hydrophobic membrane is selected from the group consisting of a polymeric microfiltration membrane, a polyvinylidene difluoride (PVDF) membrane, a positively charged nylon transfer membrane, a polytetrafluoroethylene-supported (PTFE) membrane on polypropylene or polyester, a microporous PTFE membrane, and a polypropylene membrane.

11. The fluid safety device of claim 1, wherein the sensor of at least the first, second, third, or fourth section is comprised of a cellulosic substrate infused with a colorimetric solution.

12. The fluid safety device of claim 1, wherein the colorimetric solution comprises a colorimetric substance and an alcohol, and
wherein the colorimetric substance is selected from the group consisting of phenolphthalein, p-xylenolphthalein, and thymolphthalein.

13. The fluid safety device of claim 1, wherein the sensor of at least the first, second, third, or fourth section is a blotting paper comprising a triglyceride detecting mixture, and
wherein the triglyceride detecting mixture comprises:
an HCl buffer;
an enzyme;
a dye; and
a diaphorase solution.

14. The fluid safety device of claim 1, wherein the sensor of at least the first, second, third, or fourth section is a blotting paper applied with a triglyceride detecting mixture, and
wherein the triglyceride detecting mixture comprises:
a solution comprising water, a nonionic surfactant, a detergent, a poly methyl vinyl ether, calcium chloride, Na-ATP, sucrose, and HPC (hydroxypropyl cellulose);
an enzyme;
a chromogenic substrate;
a horseradish peroxidase mixture; and
EDTA.

15. The fluid safety device of claim 1, wherein the sensor of at least the first, second, third, or fourth section is a filter paper applied with a triglyceride detecting mixture,
wherein the triglyceride detecting mixture comprises a dye solution, and
wherein the dye solution comprises a lysochrome selected from the group consisting of an amino acid staining azo dye, an aromatic compound containing an azo group, and a diazo dye.

16. The fluid safety device of claim 1, wherein the sensor of at least the first, second, third, or fourth section is comprised of an octanoyl-CoA substrate.

17. The fluid safety device of claim 1, wherein the standard color card has a chart of colors which correlate to the presence of the contaminant in a test sample, and
wherein a user can determine the presence of the contaminant in the test sample based on a color change of the reaction pad when contacted with a test sample and the standard color card.

18. The fluid safety device of claim 1, wherein the distal end further comprises a reaction pad well having a bottom surface and four sides that form an enclosure capable of holding the test sample and having an open top,
wherein the four sides are the same height as the detectors in each section,
wherein the bottom surface of the reaction pad well is integrated into an outer surface of the distal end of the fluid safety device such that the reaction pad well retains the test sample when positioned horizontally.

19. The device of claim 18, wherein the reaction pad well further comprises a retractable cover that seals the enclosure.

20. The fluid safety device of claim 19, wherein the proximal end further comprises a retractable bipodal support extension
wherein the bipodal support extension has a height such that the enclosure is level with a flat surface on which the fluid safety device is disposed, and
wherein the distal end of the fluid safety device is oriented upwards at an angle of 10-30° from the axis of the body.
